(19) United States
(12) Patent Application Publication
(10) Pub. No.: US 2009/0131312 A1
(43) Pub. Date: May 21, 2009

(54) Non-natural chemokine receptor ligands and methods of use thereof

(76) Inventors: Lawrence Blatt, San Francisco, CA (US); Scott Seiwert, Pacifica, CA (US); Hua Tan, Daly City, CA (US); Roderick Phillips, San Francisco, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/913,768

(22) PCT Filed: May 17, 2006
(86) PCT No.: PCT/US06/19233

§ 371 Date: May 23, 2008

Related U.S. Application Data
(60) Provisional application No. 60/682,544, filed on May 18, 2005.

(51) Int. Cl. A61K 38/16; C07K 14/00; C12N 15/11; C12N 15/00; A61K 31/7088; C12N 5/06; C12P 21/04; C07K 16/18

(52) U.S. Cl. 514/12; 530/324; 530/350; 536/23.5; 435/320.1; 435/325; 435/69.1; 530/387.9; 514/44

(57) ABSTRACT

The present invention provides non-natural CXCR3 ligands comprising the N-loop region of the iTAC and polynucleotide encoding such non-natural CXCR3 ligands. The invention additionally provides methods of treating fÊbrotic disorders, angiogenic disorders, and cancer. The methods generally involve administering to an individual in need thereof an effective amount of a non-natural CXCR3 ligand of the invention.

### FIELD OF THE INVENTION

The present invention is in the field of ligands for chemokine receptors, and in the treatment of fibrotic disorders, angiogenic disorders, cancer, and bacterial infections.

### BACKGROUND OF THE INVENTION

Chemokines are a family of small cytokines that are produced in inflammation and regulate leukocyte recruitment. Chemokines are capable of selectively inducing chemotaxis of leukocytes, such as neutrophils, monocytes, macrophages, eosinophils, basophils, mast cells, and lymphocytes, including T-cells and B-cells. In addition to stimulating chemotaxis, chemokines induce changes in cell shape, transient rises in the concentration of intracellular free calcium ions, granule exocytosis, integrin upregulation, formation of bioactive lipids (e.g., leukotrienes) and respiratory burst associated with leukocyte activation. Thus, chemokines play an early role in inflammatory response, causing inflammatory mediator release, chemotaxis and extravasation to sites of infection or inflammation.

Four families of chemokines have been identified and grouped according to the number and arrangement of conserved amino-terminal cysteine motifs. CC chemokines (β-chemokines) comprise adjacent cysteine residues; CXC chemokines (α-chemokines) comprise cysteine residues separated by a single, additional residue; and CX3C chemokines comprise cysteine residues separated by three additional residues.

The CXC chemokines are further divided into ELR and non-ELR chemokines, depending on the presence or absence of an additional Glu-Leu-Arg (i.e., ELR) tripeptide sequence adjacent to the CXC motif. Examples of ELR CXCs include interleukin-8 (IL-8), epithelial-derived neutrophil-activating protein (ENA), neutrophil-activating protein (NAP), and several growth-related proteins (e.g., GRO-α, β, γ). Non-ELR CXC chemokines include interferon-γ (IFN-γ)-inducible 10-kDa protein (IP-10), IFN-γ-induced monokine (MIG), IFN-inducible T-cell chemoattractant (iTAC), stromal cell-derived factor (SDF), and platelet factor 4 (PF4).

IP-10, MIG, and iTAC are potent chemoattractants for activated T-cells but not resting T-cells, B-cells or natural killer (NK) cells. Their expression appears to be upregulated in Th1-associated disorders, in response to which IFN-γ is expressed. IP-10, MIG, and iTAC expression is primarily associated with activated endothelial cells and IFN-γ-activated macrophages.

The expression of non-ELR CXC chemokines in other cells has also been reported. Specifically, IP-10 is IFN-γ-induced in monocytes, fibroblasts, astrocytes, keratinocytes, neutrophils, and endothelial cells, with expression being associated with, e.g., ulcerative colitis, atherosclerosis, sarcoidosis, tuberculoid leprosy, psoriasis, and viral meningitis (Sauty et al.; Qin et al.). MIG is IFN-γ-induced in peripheral blood mononuclear cells (PBMCs), fibroblasts, keratinocytes, endothelial cells, and PMA-stimulated monocytes. MIG expression is also associated with psoriasis. iTAC is expressed by activated monocytes and astrocytes.

The expression of these non-ELR CXC chemokines would appear to play a role in the recruitment of activated T-cells to the epithelium, likely to promote protective immunity or amplify a Th1-type immune response.

CC and CXC chemokines act through receptors which belong to a superfamily of seven transmembrane spanning G protein-coupled receptors. This family of G-protein coupled (serpentine) receptors comprises a large group of integral membrane proteins, containing seven transmembrane-spanning regions. The receptors are coupled to G proteins, which are heterotrimeric regulatory proteins capable of binding GTP and mediating signal transduction from coupled receptors, for example, by the production of intracellular mediators.

The CXC chemokine receptors 1 through 4 (CXCR1-4) bind CXC chemokines. CXCR3 (CD183) is the receptor for IP10, MIG, and iTAC. Signaling through CXCR3 induces chemotactic migration of inflammation-associated effector T cells.

### SUMMARY OF THE INVENTION

One embodiment of the invention provides a non-natural CXCR3 polypeptide receptor ligand wherein the N-loop domain is from iTAC. In a particular embodiment, the non-natural CXCR3 polypeptide receptor ligand except for the N-loop domain has a native amino acid sequence. Such non-natural ligands include, for example, the native sequence of IP-10 or MIG but with the N-loop from iTAC. In a further embodiment, the polypeptide receptor ligand except for the N-loop domain has both native and non-native amino acid sequences, wherein the non-native amino acid sequences result from substituting at a homologous position at least one native amino acid residue with a majority consensus residue.

Homologous positions are determined by first aligning the polypeptide sequences of a plurality of CXCR3 receptor ligands, or even other CXC receptor ligands (e.g., using a sequence alignment algorithm). The amino acid residues present at each aligned position are then compared at each aligned position and determined to be “identical,” “majority consensus,” or “unique” (i.e., “non-homologous”) based on the criteria described, below. Majority consensus residues are then substituted at all or some of the homologous positions.

Particular embodiments of the invention provide a non-natural CXCR3 polypeptide ligand comprising a sequence of SEQ ID NO: 3, SEQ ID NO: 6, SEQ ID NO: 9, SEQ ID NO: 10, and SEQ ID NO: 11, and variations of SEQ ID NOs: 3, 6, 9, 10, and 11, wherein positions other than 12-17 are changed to an amino acid from a homologous position in any of iTAC, IP-10 or MIG CXCR3 polypeptide ligands. For example, the position of the iTAC N-loop in relation to the N-terminus of the non-natural CXCR3 polypeptide receptor ligand may be altered by one, two, three, or even four amino acid residues. In this manner, the iTAC N-loop is located at positions 8-13, 9-14, 10-15, 11-16, 13-18, 14-19, 15-20, or 16-21 of the non-natural CXCR3 receptor ligand of the invention. The non-natural CXCR3 polypeptide ligands of the invention may also comprise native polypeptide sequences of iTAC that flank the iTAC N-loop. For example, in some embodiments, the native N-terminus of iTAC (i.e., amino acid residues 1-11 of the mature polypeptide) are present in the non-natural CXCR3 polypeptide ligands. In this example, a non-natural version of mature IP-10 or MIG could comprise as residues 1-17, the sequence representing residues 1-17 of native iTAC. Further variations are contemplated as described below.

Yet another embodiment provides a non-natural PF4 CXCR3 polypeptide receptor ligand wherein the N-loop domain is from iTAC. In one embodiment, the polypeptide receptor ligand, except for the N-loop, has a native amino acid sequence of PF4. In a particular embodiment, the sequence is that of SEQ ID NO: 13. The invention includes non-natural PF4 CXCR3 polypeptide receptor ligands having variant PF4 polypeptide sequences. In a particular embodiment, the variant polypeptide sequences comprise conservative amino acid substitutions. In another embodiment, the variant polypeptide sequences comprise amino acid residues of different CXCR3, or even different CXC ligands.

The non-natural CXCR3 polypeptide ligand of the invention may be co- or post-translationally modified, for example, by glycosylation, amidation, prenylation, farnesylation, acylation, acetylation, phosphorylation, pegylation, and the like.

In some embodiments of the invention, the non-natural CXCR3 polypeptide ligand is a mature form, lacking the signal peptide. The non-natural CXCR3 polypeptide ligand may comprise a methionine at the N-terminus.

Another embodiment of the invention provides a polynucleotide encoding the non-natural CXCR3 polypeptide receptor ligands of the invention. Such polynucleotides may encode the mature or precursor form of the non-natural CXCR3 receptor ligand of the invention, or a portion, thereof, having biological activity. In a further embodiment the polynucleotide is provided in an expression vector, operably linked to a suitable promoter. A related embodiment of the invention provides a host cell comprising a polynucleotide encoding a non-natural CXCR3 polypeptide receptor ligand.

Yet another embodiment provides a method for producing a non-natural CXCR3 ligand of the invention, the method comprising culturing the host cell comprising a polynucleotide encoding a non-natural CXCR3 ligand under conditions that favor production of the CXCR3 ligand, and isolating the CXCR3 ligand expressed in the culture.

Another embodiment of the invention provides an antibody that specifically binds a non-natural CXCR3 ligand.

The invention also provides methods for treating diseases, disorders, and/or conditions using the polypeptides and polynucleotides disclosed herein.

For example, the invention provides a method for treating a fibrotic disease in an individual. The method comprises administering to an individual suffering from a fibrotic disease an amount of a non-natural CXCR3 receptor ligand that is effective in the treatment or prophylaxis of the fibrotic disease.

Similarly, the invention provides a method for treating a fibrotic disease in an individual by administering an effective amount of a polynucleotide encoding a non-natural CXCR3 ligand. In a particular embodiment, the polynucleotide encoding the non-natural CXCR3 ligand is provided in a viral vector. The vector may comprise a polynucleotide encoding the mature CXCR3 ligand, or the precursor form with the signal peptide, or routine variations, thereof.

The fibrotic disease may be pulmonary fibrosis. In a particular aspect, the pulmonary fibrosis is idiopathic pulmonary fibrosis. In another particular embodiment, the pulmonary fibrosis is from a known etiology. The fibrotic disease may be selected from liver fibrosis, renal fibrosis, cardiac fibrosis, and scleroderma.

The invention further provides a method for reducing tumor growth in an individual having a tumor by administering to the individual an effective amount of a non-natural CXCR3 ligand and/or a polynucleotide encoding a non-natural CXCR3 ligand. The polynucleotide encoding the non-natural CXCR3 ligand may be provided in a viral vector.

The invention includes administering, with the polypeptide or polynucleotide of the invention, an effective amount of an anti-neoplastic agent, including but not limited to an alkylating agent, a nitrosourea, an antimetabolite, an antitumor antibiotic, a plant (vinca) alkaloid, a taxane, and a steroid hormone.

The methods of the invention may be used for treatment or prophylaxis in a human, typically a patient or person at risk for becoming afflicted with a disease, disorder, or condition to which the invention is related. However, the invention may also be used for treatment or prophylaxis of animals, including domestic livestock and pets.

### BRIEF DESCRIPTION OF THE DRAWING

FIG. 1 depicts a polypeptide alignment of IP-10, MIG, and iTAC. Residues that are identical in all three CXCR3 ligands at aligned positions are indicated by medium grey shading. Majority consensus residues are indicated by light grey shading. Unique (i.e., non-homologous) residues are indicated by dark grey shading. A “majority” polypeptide sequence is indicated above the aligned sequences.

FIG. 2 depicts polypeptides related to or derived from iTAC. The sequences are described in the specification.

FIG. 3 depicts polypeptides related to or derived from IP-10. The sequences are described in the specification.

FIG. 4 depicts polypeptides related to or derived from MIG. The sequences are described in the specification.

FIG. 5 depicts polypeptides related to or derived from PF4. The sequences are described in the specification.

### DETAILED DESCRIPTION OF THE INVENTION

Prior to describing the preferred embodiments of the invention, the following terms are defined. Terms that are not defined should be given their ordinary meaning in the art.

The term “polypeptide” refers to a polymer of amino acid residues (which are often referred to as “amino acids” or “residues,” to reduce verbiage). The terms “peptides,” “oligopeptides,” and “proteins” are included within the definition of polypeptide. The term “polypeptide” neither requires nor excludes co- and/or post-translational modifications, including but not limited to glycosylation, amidation, prenylation, farnesylation, acylation, acetylation, phosphorylation, pegylation, and the like. Polypeptides may include one or more amino acid analogs (e.g., non-naturally-occurring amino acids) or other functional groups, such as biotin, epitope tags, fluorescence and/or quenching groups, and the like.

The terms “polynucleotide” and “nucleic acids” refer to polymeric forms of nucleotides of any length. The polynucleotides may contain deoxyribonucleotides, ribonucleotides, and/or analogs, thereof. As used herein, polynucleotides include linear or circular double or single-stranded molecules, including but not limited to oligonucleotide primers, plasmids, expression vectors (including viral expression vectors), cosmids, artificial chromosomes, and naturally occurring chromosomes. The polynucleotides may be conjugated to other molecules, including peptides (such as biotin), fluorescence and/or quenching groups, epitope tags, and the like.

The terms “aligned position,” “identical position,” “homologous position,” majority consensus residue, and “unique” or “non-homologous position,” are defined as follows: The term “aligned position” refers to any single position in a sequence alignment (e.g. an alignment of CXCR3 polypeptide ligands). In a polypeptide sequence alignment, an aligned position is any single amino acid residue position. Where all aligned sequences have an identical residue at an aligned position, that aligned position is referred to as an “identical position.” Where a plurality of aligned polypeptide sequences have the same amino acid residue at an aligned position but one or more other aligned polypeptide sequences have a different residue at the aligned position, the aligned position is called a “homologous position.” In such cases, the amino acid residue that is most common among the aligned polypeptide sequences is called a “majority consensus residue.” In cases where there is no plurality of aligned polypeptide sequences having the same amino acid residue at an aligned position, that position is called a “unique” or “non-homologous” position (see also the definition of “consensus amino acid residue at a homologous position,” which is discussed, below).

As used herein, “conservative amino acid substitutions” and related terms refer to the replacement of one residue for another that is similar in polarity, charge, solubility, hydrophobicity, hydrophilicity, and/or amphipathic nature. Examples of “conservative amino acid substitutions” include but are not limited to: (i) aspartic acid and glutamic acid; (ii) lysine and arginine; (iii) leucine, isoleucine, and valine; (iv) glycine and alanine; (v) asparagine and glutamine; (vi) serine and threonine; and (vii) phenylalanine and tyrosine.

The term “percentage sequence identity,” with respect to another polynucleotide or polypeptide, means the percentage of bases or amino acid residues, respectively that are the same when comparing the two sequences. Percentage sequence identity can be determined in a number of different manners, which are known in the art. Programs useful for determining percentage sequence identity include BLAST, FASTA, and Smith-Waterman.

The term “non-natural CXCR3 polypeptide receptor ligand,” non-natural CXCR3 ligand,” or variations thereof, particularly where context implies the missing description, refers to a polypeptide related to a naturally-occurring CXCR3 polypeptide ligand, such as iTAC, IP-10, MIG, or PF4 but having instead a polypeptide sequence different from a naturally occurring (or “native”) CXCR3 polypeptide ligand. Such non-natural CXCR3 polypeptide ligands may be chimeras comprising polypeptide sequences, including domains, or other discrete structural and/or functional regions, from one or more naturally occurring CXCR3 polypeptide ligands. Non-natural CXCR3 ligands may comprise amino acid substitutions, additions, or deletions. Substitutions include conservative and non-conservative substitutions. The polypeptide sequences of the non-natural CXCR3 ligands of the invention may include consensus amino acid residues, as determined, e.g., by aligning a plurality of natural CXCR3 ligands.

The terms “majority consensus amino acid residue at a homologous position,” “consensus homologous amino acid residue,” or variations thereof, refer to the amino acid residue that appears most frequently at a homologous position (see above) in a polypeptide sequence alignment. The following alignment of three hypothetical polypeptide sequences further illustrates the meaning of “consensus amino acid residues at a homologous position.”

| | |
|---|---|
| Position: | 1 2 3 4 5 6 |
| Sequence A: | Gly-Gly-His-Ala-Phe-Ser |
| Sequence B: | Ala-Gly-Trp-Ile-Cys-Ser |
| Sequence C: | Ala-Lys-Phe-Val-Phe-Ser |
| Consensus: | Ala-Gly-Xaa-Xaa-Phe-Ser |

The above alignment involves three polypeptide sequences, A, B, and C. Aligned position 1 corresponds to a Gly in aligned sequence A and an Ala in aligned sequences B and C. Aligned position 1 is considered “homologous” because a plurality of the aligned polypeptide sequences (i.e., B and C) comprise the same residue at position 1. The “majority consensus” amino acid at position 1 therefore is Ala. Aligned position 2 is occupied by the amino acid residues Gly and Lys. Here, the majority consensus residue at the homologous position is a Gly since it is present at position 2 in a plurality of the aligned sequences. In the case of positions 3 and 4, no single amino acid residue appears in a plurality of the aligned sequences. Therefore, these positions are not “homologous” but rather “unique” or “non-homologous.” No consensus amino acid residue is selected to occupy positions 3 or 4; instead, the “consensus” sequence would comprise an amino acid residue that is naturally present in the sequence. Aligned position 5 corresponds to Phe in aligned sequences A and C and Cys in aligned sequence B. The consensus residue is thus Phe. Aligned position 6 corresponds to Ser in each of the aligned polypeptide sequences. Aligned position 6 is, therefore, an “identical” position.

The term “native N-loop” refers to a region of a CXCR3 polypeptide downstream (i.e., closer to the C-terminus) with respect to the characteristic C—X—C motif, which mediates chemokine binding to the CXCR3 receptor. In the case of iTAC, the native N-loop comprises six amino acid residues (Ile-Gly-Pro-Gly-Val-Lys; SEQ ID NO: 14) located at positions 12-17 of the mature iTAC chemokine.

The term “mature chemokine” refers to a chemokine polypeptide lacking the N-terminal signal peptide. As used, herein, “mature chemokines” may optionally include an N-terminal methionine, e.g., to facilitate expression of the mature polypeptide in cells or in a cell free system.

The term “precursor CXC chemokine” or reasonable variations refer to a form of a CXC chemokine having the polypeptide sequence of the signal peptide and the mature polypeptide.

The term “host cell” includes any cell that can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a “recombinant host cell”.

The terms “DNA regulatory sequences” and “regulatory elements” are used interchangeably herein, to refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The terms “transformation” and “transfection” refer to the introduction of an exogenous polynucleotide into bacterial or eukaryotic cells, respectively.

With respect to a polynucleotide sequence encoding a non-natural CXCR3 ligand, “operably linked to a promoter” means positioned such that a promoter effects its transcription or expression.

The term “construct” refers to a recombinant polynucleotide, generally recombinant DNA, generated for expression of a specific nucleotide sequence(s), or to be used in the construction of other recombinant polynucleotide sequences.

The term “binds specifically,” in the context of antibody binding, refers to high avidity and/or high affinity binding of an antibody to a specific polypeptide or fragment thereof, compared to other similar polypeptides, or fragments, thereof. Antibodies which bind specifically to a polypeptide may be capable of binding different polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to a given polypeptide with a binding affinity of at least 10⁻⁷ M, at least 10⁻⁸ M, or even at least 10⁻⁹ M, 10⁻¹⁰ M, 10¹¹ M, etc. In general, an antibody with a binding affinity of 10⁻⁶ M or less is not useful in that it will not bind an antigen at a detectable level using conventional methodology currently used.

As used herein, the terms “treatment,” “treating,” and the like, refer to affecting a desired pharmacologic and/or physiologic effect. The effect may be prophylactic in terms of completely or partially preventing a disease, disorder, condition, or symptom thereof, and/or may be therapeutic in terms of a partial or complete remedy for a disease, disorder, condition, or symptom thereof. As used herein, “treatment” encompasses (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development; and (e) relieving the disease, i.e., causing regression of the disease.

The terms “individual,” “subject,” and “patient” are used interchangeably herein, to refer to a mammal, which include by way of example humans, primates, bovines, ovines, porcines, canines, felines, equines, and donkeys.

The term “therapeutically effective amount” refers to an amount of a therapeutic agent, or a rate of delivery of a therapeutic agent, effective to affect an intended or desired therapeutic effect. The precise desired therapeutic effect will vary according to the condition to be treated, the formulation to be administered, and a variety of other factors that are appreciated by those of ordinary skill in the art.

The terms “fibrotic condition,” “fibrotic disease,” and “fibrotic disorder” are used interchangeably to refer to a disease, disorder, or condition that is amenable to treatment by administration of a compound having anti-fibrotic activity. Fibrotic disorders include, but are not limited to, pulmonary fibrosis, including idiopathic pulmonary fibrosis (IPF) and pulmonary fibrosis from a known etiology, liver fibrosis, and renal fibrosis. Other exemplary fibrotic conditions include musculoskeletal fibrosis, cardiac fibrosis, post-surgical adhesions, scleroderma, glaucoma, and skin lesions such as keloids.

The terms “cancer,” “neoplasm,” and “tumor,” are used interchangeably herein to refer to cells which exhibit relatively autonomous growth, so that they exhibit an aberrant growth phenotype characterized by a significant loss of control of cell proliferation. Cancerous cells can be benign or malignant.

The term “chemotherapeutic agent” or “chemotherapeutic” (or “chemotherapy”, in the case of treatment with a chemotherapeutic agent) encompasses any non-proteinaceous (i.e., non-peptidic) chemical compound useful in the treatment of cancer. Examples of chemotherapeutic agents are disclosed in International Patent Application WO 2005/016241, which is, herein, incorporated by reference.

The term “biological response modifier” refers to any proteinaceous (i.e., peptidic) molecule or any non-proteinaceous (i.e., non-peptidic) molecule capable of elaborating or altering a biological response relevant to the treatment of cancer. Examples of biological response modifiers are disclosed in International Patent Application WO 2005/016241, which is, herein, incorporated by reference.

As used herein, the terms “Type I interferon receptor agonist,” “Type II interferon receptor agonist,” and “Type III interferon receptor agonist” refer to any naturally occurring or non-naturally occurring ligand of human Type I, Type II, or Type III interferon receptor, respectively, which binds to and causes signal transduction via the receptor. Examples of Type I, Type II, and Type III interferon receptor agonists are disclosed in International Patent Application WO 2005/016241, which is, herein, incorporated by reference.

While the invention is described in particular embodiments, it is understood that the invention is not limited to the particular embodiments, nor is the terminology used to described the particular embodiments intended to be limiting.

Unless defined otherwise, all technical and scientific terms used herein have the meaning as commonly understood by one of ordinary skill in the art to which it pertains. All publications mentioned herein are incorporated herein by reference to disclose and describe the methods and/or materials in connection with which the publications are cited.

Finally, it must be noted that as used herein and in the appended claims, the singular forms “a,” “and,” and “the” include plural referents unless the context clearly dictates otherwise. Thus, for example, reference to “a non-natural CXCR3 ligand” includes a plurality of such ligands and reference to “the formulation” includes reference to one or more formulations and equivalents thereof known to those skilled in the art, and so forth.

The present invention provides non-natural CXCR3 polypeptide receptor ligands, polynucleotides encoding such ligands, as well as compositions, formulations, and methods of use, thereof. A particular feature of the non-natural CXCR3 ligands is that they contain the native N-loop polypeptide sequence of the CXCR3 ligand, iTAC. The N-loop of iTAC is associated with the chemokine's relatively high affinity for the CXCR3 receptor.

The native iTAC N-loop will improve the specificity or affinity of different natural or non-natural CXCR3 ligands for CXCR3 receptors, which will yield more potent versions of such CXCR3 ligands. In this manner, the invention provides high affinity and/or specificity versions of IP-10, MIG, PF4, or other CXCR3 ligands, which comprise the native iTAC N-loop. The invention also provides high affinity and/or specificity variants and chimeric forms of CXCR3 receptor ligands having the native iTAC N-loop, some of which are described, herein. Such high affinity and/or specificity receptors ligands are useful for blocking angiogenesis, inducing Th-1-mediated immune responses, and enhancing all the beneficial effects associated with CXCR3 ligand expression.

The iTAC N-loop sequence (Ile-Gly-Pro-Gly-Val-Lys; SEQ ID NO: 14) is positioned in the non-natural CXCR3 ligand in substantially the same location as in the polypeptide sequence of naturally occurring iTAC, i.e., between positions 12-17 of the mature CXCR3 ligand polypeptide sequence. These positions are downstream (C-terminal) with respect to the characteristic C—X—C motif. However, the relative position of the iTAC N-loop in the non-natural CXCR3 polypeptide may be varied such that the loop is more or less proximal to the N-terminus of the non-natural CXCR3 ligand. For example, the iTAC N-loop could be positioned at amino acid residues 8-13, 9-14, 10-15, 11-16, 13-18, 14-19, 15-20, or 16-21 of the non-natural CXCR3 receptor ligand of the invention. Such repositioning may be desirable to conform with the position of the endogenous N-loop of the CXCR3 polypeptide from which the amino acid residues flanking the iTAC N-loop in the non-natural CXCR3 ligand are derived.

In addition to the iTAC N-loop polypeptide sequence of Ile-Gly-Pro-Gly-Val-Lys (SEQ ID NO: 14), the non-natural CXCR3 ligand of the invention may include additional amino acid residues derived from iTAC. In one example, the non-natural CXCR3 ligand of the invention comprises the polypeptide sequence, Phe-Pro-Met-Phe-Lys-Arg-Gly-Arg-Cys-Leu-Cys-Ile-Gly-Pro-Gly-Val-Lys (SEQ ID NO: 15) as the first 17 amino acids, of which all are from native iTAC (i.e., residues 1-17) including the N-loop at positions 12-17 (italicized). This sequence would thus replace the corresponding N-terminal through N-loop sequence of the non-natural CXCR3 ligand of the invention derived from a CXCR3 ligand other than iTAC. In other embodiments, a methionine residue may be present at position 1 for the mature form of the ligand.

Other embodiments of the non-natural CXCR3 ligands of the invention comprise the native iTAC N-loop along with only a portion of the N-terminal flanking amino acid residues of iTAC, for example, residues 2-17, 3-17, 4-17, 5-17, 6-17, 7-17, 8-17, 9-17, 10-17, or 11-17. Other embodiments of the non-natural CXCR3 ligands of the invention comprise iTAC amino acid residues that are C-terminal with respect to the N-loop. Such residues include but are not limited to amino acid residues 18, 19, 20, 21, 22, 23, and residues flanking the N-loop in iTAC. The C-terminal residues from iTAC may be present in the non-iTAC non-natural CXCR3 ligands of the invention in addition to or instead of the aforementioned N-terminal residues from iTAC.

As was the case with the iTAC N-loop, the relative position of the iTAC N-loop, along with flanking N-terminal or C-terminal residues may be varied such that the loop is more or less proximal to the N-terminus of the non-natural CXCR3 ligand. Of course, one skilled in the art will recognize that where the non-natural CXCR3 ligand of the invention comprises the complete N-terminal polypeptide sequence of the mature iTAC ligand, i.e., residue 1-17 or more, shifting the position of the N-loop relative to the non-natural CXCR3 ligand may be impractical.

Unless context provides otherwise, any of the non-natural CXCR3 ligands described, herein, may comprise iTAC N-loop-flanking N-terminal and/or C-terminal residues as replacements for the native amino acid at aligned positions of the non-iTAC CXCR3 ligands.

The non-natural CXCR3 polypeptide ligand may additionally comprises sequence derived from one or more CXC ligands. Preferably, these ligands are non-ELR CXCR3 ligands, such as iTAC, MIG, IP-10, or PF4, with the proviso that the resulting polypeptide sequence is not identical to that of a native CXCR3 ligand, including iTAC. In some cases, the additional polypeptide sequences are chimeric, being derived from a plurality of non-ELR CXCR3 ligands. Chimeric non-natural CXCR3 ligands are described in International Patent Application WO 2005/016241, which is, herein, incorporated by reference.

The non-natural CXCR3 polypeptide ligand of the invention alternatively or additionally comprises majority consensus amino acid residues. Majority consensus amino acid residues are determined by aligning a plurality of CXCR3 ligands, e.g., using an algorithm such as BLAST, FASTA, or Smith Waterman, which are known in the art, and comparing the amino acid residues present at each aligned position.

In a particular embodiment, the non-natural CXCR3 ligand comprises consensus residues only at homologous positions, as defined, above. In some embodiments of the invention all residues at homologous positions are replaced by consensus residues. In other embodiments, only a subset of the homologous residues are replaced with consensus residues, while others are not replaced. In the latter case, the positions not replaced with consensus residues (with the exception of the iTAC N-loop) may comprise any amino acid residues present at the aligned positions.

In some embodiments of the invention, the “unique” residues in the non-natural CXCR3 ligand are from the same native CXCR3 ligand. In other embodiments, the unique residues are derived from a plurality of native CXCR3 ligands. In particular embodiments, the unique residues are derived from IP-10, MIG, iTAC, or combinations, thereof. In further embodiments, the unique residues are derived from other CXC ligands, not limited to those described, herein. In all cases, the non-natural CXCR3 ligands of the invention comprise a native iTAC N-loop.

The invention further provides variants of the above-described non-natural CXCR3 ligands that comprise amino acid substitutions, deletions, and insertions, except in the native iTAC N-loop sequence and provided that such variations do not eliminate the ability of the ligand to bind to the CXCR3 (CD183) receptor and induce chemotactic migration of inflammation-associated effector T cells. Such variants may include conservative substitutions and/or non-conservative substitutions, as defined above and through-out the specification. Examples of conservative substitutions are described, above.

### Non-Natural CXCR3 Ligands

Examples of polypeptides of the invention are provided in the accompanying Figures and in the Sequence Listing. These sequences are derived from the amino acid sequence of native human IP-10, MIG, and iTAC that are found in GenBank. For example, IP-10 is found in GenBank as Accession Nos. PO2778, NP_{—}001556, and 1312356A. In these sequences, amino acid residues 1-21 are a signal sequence, while amino acids residues 22-98 are the mature region of IP-10. MIG is found in GenBank as Accession Nos. NP_{—}002407 and Q07325. In these sequences, amino acids 1-22 are a signal sequence, and mature MIG is amino acids 23-125. iTAC is found in GenBank under Accession Nos. Q14625 and AAD38867. In these sequences, amino acids 1-21 are a signal sequence, and mature iTAC is amino acids 22-94. Note that the sequences described herein are those of mature CXC chemokines and the first amino acid residue of the mature polypeptides is designated position 1.

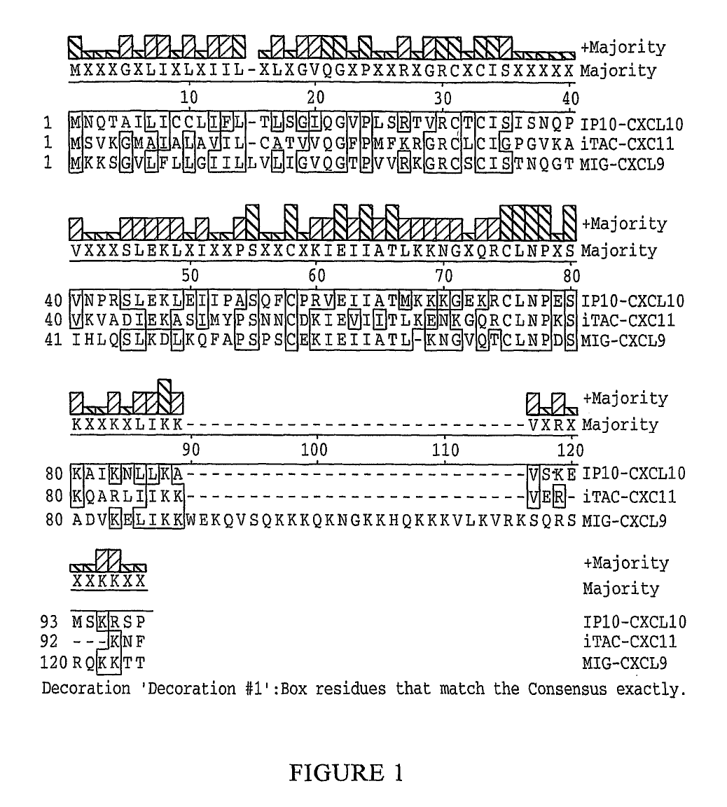

To illustrate the difference between identical, majority consensus, and unique residues, a LAZERGENE 6 alignment of IP-10, MIG, and iTAC is shown in FIG. 1. Amino acid positions that are occupied by identical residues in all three CXCR3 ligands are indicated by medium grey shading. This identical residue is also identified in the “Majority” polypeptide sequence, which is located above the alignment.

Majority consensus residues at homologous positions are indicated by light grey shading and the most frequently appearing (i.e., consensus) residue is indicated in the “Majority” polypeptide sequence.

Unique residues at a homologous position are indicated by dark grey shading. An “X” (i.e., “Xaa”) appears in the majority polypeptide sequence to indicate that such residues are not changed to consensus residues in the non-natural CXCR3 polypeptide ligands of the invention. Instead, such residues are selected from any of the residues present at that aligned position in any of the aligned sequences.

The following Table summarizes the positions of identical (I), homologous (H), and unique (U) amino acid residue positions, based on the alignment of the iTAC, IP-10, and MIG polypeptides, which is shown in FIG. 1. The Table also correlates these amino acid residue positions in the precursor forms of the polypeptides to the corresponding positions in the mature forms of the polypeptides. The gap in the alignment at positions 90-116 has been excluded from the Table.

| Position | Corresp. position | | | |
|---|---|---|---|---|
| in FIG. 1 | in mature | I | H | U |
| 23 | 1 | | | X |
| 24 | 2 | X | | |
| 25 | 3 | | | X |
| 26 | 4 | | | X |
| 27 | 5 | | X | |
| 28 | 6 | | | X |
| 29 | 7 | | X | |
| 30 | 8 | X | | |
| 31 | 9 | X | | |
| 32 | 10 | | | X |
| 33 | 11 | X | | |
| 34 | 12 | X | | |
| 35 | 13 | | X | |
| 36 | 14 | | | X |
| 37 | 15 | | | X |
| 38 | 16 | | | X |
| 39 | 17 | | | X |
| 40 | 18 | | | X |
| 41 | 19 | | X | |
| 42 | 20 | | | X |
| 43 | 21 | | | X |
| 44 | 22 | | | X |
| 45 | 23 | | X | |
| 46 | 24 | | X | |
| 47 | 25 | | X | |
| 48 | 26 | | X | |
| 49 | 27 | | X | |
| 50 | 28 | | | X |
| 51 | 29 | | X | |
| 52 | 30 | | | X |
| 53 | 31 | | | X |
| 54 | 32 | | X | |
| 55 | 33 | X | | |
| 56 | 34 | | | X |
| 57 | 35 | | | X |
| 58 | 36 | X | | |
| 59 | 37 | | | X |
| 60 | 38 | | X | |
| 61 | 39 | | X | |
| 62 | 40 | X | | |
| 63 | 41 | | X | |
| 64 | 42 | X | | |
| 65 | 43 | | X | |
| 66 | 44 | X | | |
| 67 | 45 | | X | |
| 68 | 46 | | X | |
| 69 | 47 | | X | |
| 70 | 48 | | X | |
| 71 | 49 | | X | |
| 72 | 50 | | | X |
| 73 | 51 | | X | |
| 74 | 52 | | X | |
| 75 | 53 | X | | |
| 76 | 54 | X | | |
| 77 | 55 | X | | |
| 78 | 56 | X | | |
| 79 | 57 | | | X |
| 80 | 58 | X | | |
| 81 | 59 | | X | |
| 82 | 60 | | | X |
| 83 | 61 | | | X |
| 84 | 62 | | X | |
| 85 | 63 | | | X |
| 86 | 64 | | X | |
| 87 | 65 | | X | |
| 88 | 66 | X | | |
| 89 | 67 | | X | |
| 117 | 95 | | X | |
| 118 | 96 | | | X |
| 119 | 97 | | X | |
| 120 | 98 | | | X |
| 121 | 99 | | | X |
| 122 | 100 | | | X |
| 123 | 101 | | X | |
| 124 | 102 | | X | |
| 125 | 103 | | | X |
| 126 | 104 | | | X |

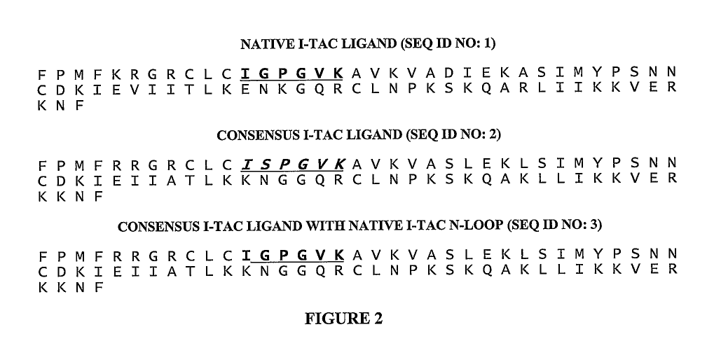

FIGS. 2-5 shows the relationships between some of the polypeptides of the invention and those previously described. All the sequences shown in FIGS. 2-5 are mature forms (i.e., they lack a signal peptide).

FIG. 2 shows three polypeptides related to or derived from iTAC. SEQ ID NO: 1 is native iTAC. The native iTAC N-loop is underlined. SEQ ID NO: 2 is “consensus iTAC, in which the amino acid residues of native iTAC have been replaced with the majority consensus residues present at homologous position in iTAC, MIG, and IP-10 (referring to FIG. 1). Consensus iTAC comprises an amino acid substitution in the N-loop region, wherein the Gly at the second amino acid position of the native iTAC N-loop is replaced with a consensus Ser residue (underlined and italicized). SEQ ID NO: 3 is an example of a non-natural CXCR3 ligand of the invention. SEQ ID NO: 3 is similar to SEQ ID NO: 2, except that it comprises the native iTAC N-loop (underlined).

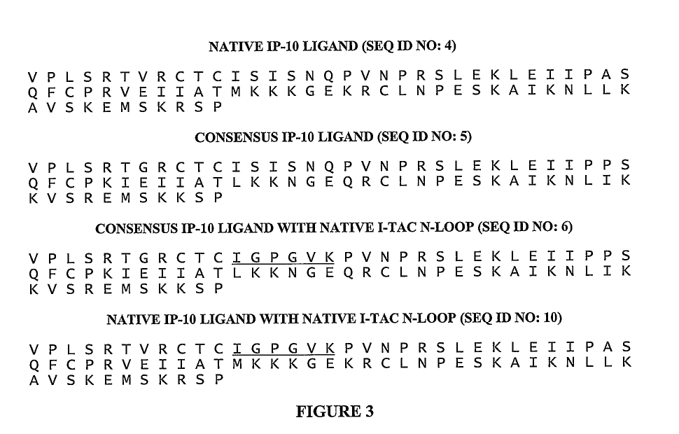

FIG. 3 shows four polypeptides related to or derived from IP-10. SEQ ID NO: 4 is native IP-10. SEQ ID NO: 5 is “consensus IP-10, in which the amino acid residues of native IP-10 have been replaced with consensus residues present at homologous position in iTAC, MIG, and IP-10 (referring to FIG. 1). SEQ ID NOs: 6 and 10 are examples of a non-natural CXCR3 ligand of the invention. In SEQ ID NO: 6, the native iTAC N-loop (underlined) has been inserted into the consensus IP-10 polypeptide sequence. In SEQ ID NO: 10, the native iTAC N-loop (underlined) has been inserted into the native IP-10 polypeptide sequence.

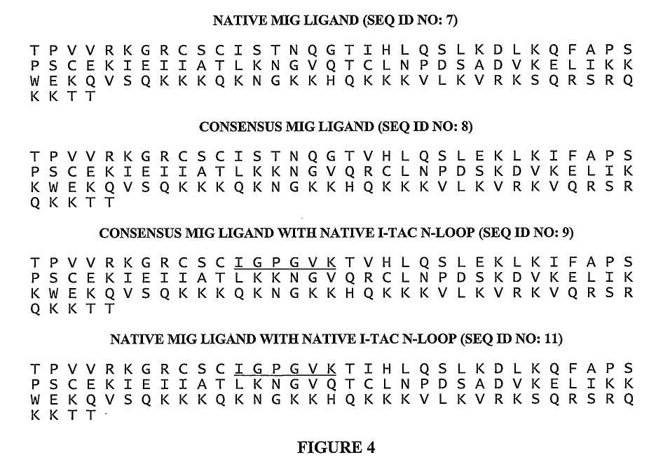

FIG. 4 shows four polypeptides related to or derived from MIG. SEQ ID NO: 7 is native MIG. SEQ ID NO: 8 is “consensus MIG,” in which the amino acid residues of native MIG have been replaced with consensus residues present at homologous positions in iTAC, MIG, and IP-10 (referring to FIG. 1). SEQ ID NOs: 9 and 11 are examples of non-natural CXCR3 ligands of the invention. In SEQ ID NO: 9, the native iTAC N-loop (underlined) has been inserted into the consensus MIG polypeptide sequence. In SEQ ID NO: 11, the native iTAC N-loop (underlined) has been inserted into the native MIG polypeptide sequence.

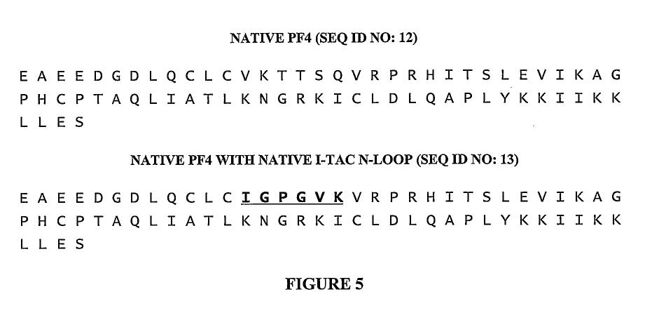

FIG. 5 shows two polypeptides related to or derived from PF4. SEQ ID NO: 12 is native PF4, a CXCR3 ligand related to iTAC, MIG, and IP-10 but having reduced structural similarity to these ligands. SEQ ID NO: 13 is an example of a non-natural CXCR3 ligand of the invention, in which the native iTAC N-loop (underlined) has been inserted into the PF4 polypeptide sequence.

As noted, above, FIGS. 2-5 show polypeptide sequences of mature CXCR3 ligands, which lack signal peptides. Since the signal peptides are proteolitically co- or post-translationally removed from precursor polypeptides having signal peptides, the mature polypeptide sequence of CXCR3 ligands generally lack N-terminal Methionine residues. However, the invention includes a mature form of any of the non-natural CXCR3 ligands, that are described or enabled by the instant disclosure, and that further comprise an N-terminal Methionine, which may be added to allow the expression of the mature form of the ligand, without the need for co- or post-translational processing.

### Polypeptide Modifications

In some embodiments, a non-natural CXCR3 polypeptide ligand includes one or more modifications. Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation (addition of a polyethylene glycol moiety); and the like. In one embodiment, the invention contemplates the use of non-natural CXCR3 ligand variants with one or more non-naturally occurring glycosylation and/or pegylation sites that are engineered to provide glycosyl- and/or PEG-derivatized polypeptides with reduced serum clearance. Thus, the invention includes PEGylated non-natural CXCR3 ligands. Also included are modifications by glycosylation, phosphorylation, sulfonation/sulfation, amidation, acylation, acetylation, methylation, hydroxylation, ADP-ribosylation, carboxylation, adenylation, ubiquitination, farnesylation, prenylation, metal addition, maturation, proteolytic cleavage, and other known but not listed additions and/or subtractions to a polypeptide.

In some embodiments, the non-natural CXCR3 ligand polypeptide is a fusion polypeptide comprising a non-natural CXCR3 ligand polypeptide and a heterologous polypeptide (e.g., a fusion partner). Suitable fusion partners include peptides and polypeptides that confer enhanced stability in vivo, facilitate isolation and/or purification, provide a detectable signal, provides for multimerization, or direct appropriate co- and/or post-translational processing (e.g., a signal peptide). A fusion protein may also comprise an amino acid sequence that provides for secretion of the fusion protein from the cell (See, e.g., U.S. Pat. No. 5,712,113) or provide a protease cleavage site. Examples of fusion partners and modifications to polypeptides that are useful for practicing the invention are also disclosed in International Patent Application WO 2005/016241, which is, herein, incorporated by reference.

### Polynucleotides, Vectors, and Host Cells

The present invention further provides a polynucleotide comprising a nucleotide sequence that encodes a non-natural CXCR3 ligand of the invention, vectors comprising such polynucleotides. A polynucleotide is useful for generating a subject expression vector and genetically modified host cells, which are useful for producing a non-natural CXCR3 ligand of the invention.

Thus, the subject invention provides nucleic acids comprising a nucleotide sequence encoding a non-natural CXCR3 ligand, and nucleic acids having substantial nucleotide sequence identity to such nucleic acids (e.g., homologs). In many embodiments, a subject nucleic acid comprises a nucleotide sequence that encodes a non-natural CXCR3 ligand and that has at least about 75%, at least about 80%, at least about 85%, at least about 90%, at least about 95%, at least about 98%, or at least about 99%, or more, nucleotide sequence identity with a nucleotide sequence encoding a non-natural CXCR3 ligand (e.g., with the CXCR3 coding sequence), or the complementary sequence thereof. Algorithms for determining sequence identity are known in the art and some are described, herein.

Also provided are nucleic acids that hybridize to the above-described nucleic acids under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

Non-natural CXCR3 polynucleotides and polynucleotide derivatives are also disclosed in International Patent Application WO 2005/016241, which is, herein, incorporated by reference.

The invention also includes a viral vector comprising a polynucleotide encoding a non-natural CXCR3 polypeptide ligand. Viral vectors for use in gene delivery include but are not limited to, retrovirus vectors (including lentivirus vectors), adenovirus vectors, adeno-associated virus vectors, herpesvirus vectors, and poxvirus vectors. Many other viruses have been shown to be capable of expressing genes-of-interest in cells, and the construction of such recombinant viral vectors does not constitute part of the invention.

Criteria for selecting viral vectors include but are not limited to, the cell-type-specificity of the virus; the level of expression desired and the level of expression possible using a particular virus vector; the tendency of a particular viral vector to cause lysis, apoptosis, or other forms of cell death; ease of production of sufficient quantities and qualities of viral vector; the extent of immune response to a particular viral vector, which may also vary among patients; and the relative abilities of particular viral vectors to produce properly folded, post-translationally-modified, and active pleiotrophin. Viral vectors, as well as many of the advantages and disadvantages of particular viral vectors, are well-known in the art and do not constitute part of the invention.

### Preparation of a Non-Natural CXCR3 Polypeptide Ligand

A subject non-natural CXCR3 ligand is prepared using any known method, including chemical synthesis methods, production by standard recombinant techniques, and combinations thereof. For example, a non-natural CXCR3 ligand can be synthesized using an automated solid-phase tert-butyloxycarbonyl and benzyl protection strategy. A non-natural CXCR3 ligand can be synthesized by native chemical ligation, e.g., fragments of from about 15 to about 40 amino acids in length (e.g., fragments of from about 15 to about 20, from about 20 to about 25, from about 25 to about 30, from about 30 to about 35, or from about 35 to about 40 amino acids in length) can be synthesized using standard methods of chemical synthesis, and the fragments ligated, using a process as described in Dawson, et al. (1994) Science 266:776-779. The purity of synthesized polypeptides may be assessed by reverse-phase HPLC and isoelectric focusing. The primary structures of the ligands may be verified by Edman sequencing methods.

In many embodiments, an expression vector comprising a polynucleotide sequence that encodes a non-natural CXCR3 ligand is prepared, using conventional methods, and is introduced into a host cell. The expression vector provides for production of the non-natural CXCR3 ligand in the host cell.

Thus, the present invention provides a method for producing a non-natural CXCR3 ligand, the method comprising culturing a host cell, which host cell comprises an expression vector that includes a polynucleotide sequence that encodes a CXCR3 ligand, under conditions that favor production of the non-natural CXCR3 ligand by the host cell; and isolating the non-natural CXCR3 ligand from the culture (e.g., from a host cell lysate and/or from the culture medium). The method may be carried out using a eukaryotic cell or a prokaryotic cell.

The polypeptides may be expressed in prokaryotes or eukaryotes in accordance with conventional ways, depending upon the purpose for expression. Examples of particular methods for expressing the non-native CXCR3 polypeptide of the invention are disclosed in International Patent Application WO 2005/016241, which is, herein, incorporated by reference.

The present invention also provides compositions comprising a non-natural CXCR3 ligand. A CXCR3 ligand will in many embodiments be pure, e.g., at least about 90% pure (free from non-CXCR3 ligand polypeptides and/or other macromolecules), at least about 95% pure, at least about 98% pure, or at least about 99% pure, or greater than 99% pure.

A subject CXCR3 ligand composition comprises, in addition to a CXCR3 ligand, one or more of a buffer, a salt, a pH adjuster, a solubilizing agent, a chelating agent, a detergent, a non-ionic detergent, a protease inhibitor, an adjuvant, etc.

In some embodiments, a subject composition comprises a subject non-natural CXCR3 ligand; and pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Antibody Compositions

Also provided are antibodies that bind specifically to a non-natural CXCR3 ligand polypeptide. Suitable antibodies are obtained by immunizing a host animal with peptides comprising all or a portion of the non-natural CXCR3 ligand. Suitable host animals include mice, rats, rabbits, sheep, goats, hamsters, and other animals used for antibody production. Antibodies are produced, screened, and isolated by standard methods well known in the art. The antibodies may, additionally, be purified or fractionated.

Also provided are compositions comprising a subject antibody. A subject antibody composition comprises, in addition to a subject antibody, one or more of a buffer, a salt, a pH adjuster, a solubilizing agent, a chelating agent, a detergent, a non-ionic detergent, a protease inhibitor, etc.

The immunogen used to immunize an animal for the production of antibodies may comprise a precursor or mature form of any of the non-natural CXCR3 ligands of the invention, or fragments and derivatives thereof. Typical immunogens comprise all or a part of the protein, where these residues contain the post-translation modifications found on the native target protein, or the cites of such modifications. Immunogens are produced in a variety of ways known in the art, e.g., expression of cloned genes using conventional recombinant methods, chemical synthesis of non-natural CXCR3 ligand polypeptides, etc.

In preferred embodiments of the invention, an immunogen comprises a portion of a non-natural CXCR3 polypeptide ligand sequence that is different, at least one position, from native CXCR3 ligands. For example, the immunogen could comprise the iTAC N-loop sequence along with flanking sequences derived from another CXCR3 ligand. Such immunogens may be 15, 20, 25, or more amino acid residues in length, although length is not critical to the invention.

Methods for preparing the antibodies of the invention, and the type of antibodies that are included in the invention, are further disclosed in International Patent Application WO 2005/016241, which is, herein, incorporated by reference. It will be understood that an immunogen may be fused or attached to a hapten or carrier, and may be delivered to an animal along with an adjuvant, such as alum, dextran, sulfate, large polymeric anions, oil and water emulsions, e.g. Freund's adjuvant, Freund's complete adjuvant, and the like.

### Methods of Use

CXCR3 ligands are associated with fibrotic disorders, including but not limited to collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy), and the like), and the like.

The present invention provides methods for treating a fibrotic disorder in an individual having a fibrotic disorder or at risk for developing a fibrotic disorder. The method generally involves administering an effective amount of a non-natural CXCR3 ligand of the invention The methods provide for treatment of fibrotic diseases, including those affecting the lung such as idiopathic pulmonary fibrosis, pulmonary fibrosis from a known etiology, liver fibrosis or cirrhosis, cardiac and renal fibrosis. The etiology may be due to any acute or chronic insult including toxic, metabolic, genetic and infectious agents. The use of related but distinct non-natural CXCR3 ligands is disclosed in International Patent Application WO 2005/016241, which is, herein, incorporated by reference.

In some embodiments, an effective amount of a non-natural CXCR3 ligand is an amount that, when administered to an individual having a fibrotic disorder, is effective to reduce fibrosis or reduce the rate of progression of fibrosis by at least about 10%, at least about 15%, at least about 20%, at least about 25%, at least about 30%, at least about 35%, at least about 40%, at least about 45%, or at least about 50%, or more, compared with the degree of fibrosis in the individual prior to treatment or compared to the rate of progression of fibrosis that would have been experienced by the patient in the absence of treatment with the non-natural CXCR3 ligand.

In some embodiments, an effective amount of a non-natural CXCR3 ligand is an amount that, when administered to an individual having a fibrotic disorder, is effective to increase, or to reduce the rate of deterioration of, at least one function of the organ affected by fibrosis (e.g., lung, liver, kidney, etc.) by at least about 10%, at least about 15%, at least about 20%, at least about 25%, at least about 30%, at least about 35%, at least about 40%, at least about 45%, or at least about 50%, or more, compared to the basal level of organ function in the individual prior to treatment or compared to the rate of deterioration in organ function that would have been experienced by the individual in the absence of treatment with the non-natural CXCR3 ligand.

Methods of measuring the extent of fibrosis in a given organ, and methods of measuring the function of any given organ, are well known in the art.

### Combination Therapies

In some embodiments, the present invention provides combination therapies for the treatment of a fibrotic disorder. Accordingly, the present invention provides a method of treating a fibrotic disorder, generally involving administering a non-natural CXCR3 ligand in combination therapy with a second therapeutic agent. Suitable second therapeutic agents include, but are not limited to, a Type I interferon receptor agonist, a Type III interferon receptor agonist, a Type II interferon receptor agonist, pirfenidone or a pirfenidone analog, a TNF antagonist, a TGF-β antagonist, an endothelin receptor antagonist, a stress-activated protein kinase inhibitor, etc. Combination therapies suitable for use with the compositions of the invention are disclosed in International Patent Application WO 2005/016241, which is, herein, incorporated by reference.

### Gene Therapy

An alternative to administering non-natural CXCR3 polypeptide ligands is to administer polynucleotide encoding such ligands to target tissues or cells (i.e., the cells affected by a particular disease to be treated or prevented). Numerous viral and non-viral expression are known in the art and have been used to deliver genes to target cells and tissues. Non-viral vectors include a variety of expression vectors, which can be delivered to cells of an individual via transfection, transduction, ballistic delivery, and the like. Viral vectors include retroviruses, such as lentiviruses, herpesviruses, poxviruses, adenoviruses and adeno-associated viruses, and vectors based on other recombinant virus genomes.

In the case of the non-natural CXCR3 polypeptide ligands, such vectors will express either the precursor form of the polypeptide, which includes the signal peptide, or the mature form of the peptide, which lacks the signal peptide. Delivering the precursor form of the non-natural CXCR3 polypeptide ligand is more likely to promote appropriate co- and post-translational processing resulting because the signal peptide will direct the nascent polypeptide chain through the correct processing steps in the cell. The signal peptide is then cleaved by cellular enzymes.

Alternatively, the mature for of the non-natural CXCR3 polypeptide ligand may be delivered to target cells. In this case, the vector used to deliver the polypeptide will encode a polypeptide comprising an N-terminal Met before the polypeptide sequence of the mature non-natural CXCR3 polypeptide ligand.

### Dosages, Formulations, and Routes of Administration

A non-natural CXCR3 ligand, optionally in combination with one or more additional therapeutic agents, is administered to an individual in need thereof in a formulation. A wide variety of pharmaceutically acceptable excipients are known in the art and need not be discussed in herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, intravenous, subcutaneous, intramuscular, intratumoral, transdermal, intratracheal, etc., administration. Administration of non-natural CXCR3 polypeptide ligands is disclosed in International Patent Application WO 2005/016241, which is, herein, incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EXAMPLES

### Example 1

### Animal Model for Tumorigenesis

In some embodiments, a non-natural CXCR3 ligand is utilized to inhibit tumor growth in a non-human animal model for tumorigenesis. Any non-human animal model of tumorigenesis is suitable for use. An exemplary model is discussed in U.S. Pat. No. 6,491,906. The model provides for an assessment of tumorigenesis, spontaneous metastasis and experimental lung colonization. A human non-small cell lung carcinoma (NSCLC) cell line is used. Either intact NSCLC tumors or cell lines may be used. Tumor growth is assessed by tumor size and mass, while spontaneous metastasis and lung colonization (experimental metastasis) is determined by histopathologic analysis of the lungs. In this system, a non-natural CXCR3 ligand can be used as a positive control for tumor growth inhibition activity. In addition, the system can be used to screen for agonists or antagonists of non-natural CXCR3 ligand activity.

The human NSCLC/SCID mouse model particularly involves the use of SCID mice of between the ages of 4 to 6 weeks. SCID mice should only be used if their serum Ig is <1 μg/ml. Human NSCLC/SCID mice chimera receive 20 μl of anti-asialo GM1 (aASGM1; Wako Chemicals, Dallas Tex.) by tail vein 24 hours prior to tumor implantation. This therapy removes host-derived NK cells.

Using intact human NSCLC, 1 mm³ specimens (grossly devoid of necrosis and weighed) are placed subcutaneously into the bilateral flank regions of a cohort group of SCID mice. Using the NSCLC cell lines (Calu-6, A549, Calu-1, and Calu-3), semiconfluent grown tumor cells are harvested and a cohort group of SCID are given 10⁶ cells and 5×10⁵ cells in 100 μl of PBS injected into bilateral flank regions and tail vein, respectively. At least one group of SCID mice form the treatment group and are administered a subject non-natural CXCR3 ligand. All mice are monitored daily for both evidence of illness and measurement of tumor size by digital engineers calipers.

Animals are sacrificed on a weekly basis for 16 weeks or sooner if the tumor size reaches 3 cm or the animals appear ill. Animals that appear ill are sacrificed, necropsy performed, and excluded from the study if their illness is for reasons other than tumor burden. At time of sacrifice, tumors in the subcutaneous location are measured and weighed. The experimental lung tumor colonization or spontaneous lung metastasis is then determined.

The administration of a subject non-natural CXCR3 ligand will have a significant attenuating effect on tumor growth within SCID mice. Thus, the tumor growth inhibition activity of non-natural CXCR3 ligand can be used as a positive control for comparison to the tumor growth inhibition activity of a candidate anticancer compound. Alternatively, the system can be used to evaluate the activity of a candidate agonist or antagonist of the tumor growth inhibition activity of non-natural CXCR3 ligand.

### Example 2

### Endothelial Cell Chemotaxis Assay

A subject non-natural CXCR3 ligand can also be used in the evaluation of candidate agents for endothelial cell chemotactic activity. An endothelial cell chemotaxis assay is performed in 48-well, blind well chemotaxis chambers (Nucleopore Corp., Maryland). Nucleopore chemotaxis membranes (5 micron pore size) are prepared by soaking them sequentially in 3% acetic acid overnight and for 2 hours in 0.1 mg/ml gelatin. Membranes are rinsed in sterile water, dried under sterile air, and stored at room temperature for up to 1 month. Bovine adrenal gland capillary endothelial cells (BCE), maintained in gelatin-coated flasks in DME with 10% FBS are used as the target cells. Twenty four hours before use, BCE are starved in DME with 0.1% BSA. Twenty five microliters of cells, suspended at a concentration of 1×10⁶ cells per ml in DME with 0.1% BSA are dispensed into each of the bottom wells. A chemotaxis membrane is positioned atop the bottom wells, chambers are sealed, inverted, and incubated for 2 hours to allow cells to adhere to the membrane. Chambers are then reinverted, 50 ml test media is dispensed into the top wells and reincubated for an additional 2 hours. Membranes are then fixed and stained with Diff-Quick staining kit (American Scientific Products) to enumerate membrane-bound cells, and cells that had migrated through the membrane to the opposite surface are counted.

The presence of a subject non-natural CXCR3 ligand in the test media will induce cell migration across the chamber membrane. Thus, a subject non-natural CXCR3 ligand can be used as a positive control in the system. Alternatively, the system can be used to evaluate candidate agonists or antagonists of the chemotactic activity of a non-natural CXCR3 ligand.

### Example 3

### In Vivo Angiogenesis Assay

In addition, a non-natural CXCR3 ligand can be used in the evaluation of the anti-angiogenic activity of a candidate agent in an in vivo model of angiogenesis. The well-characterized corneal micropocket model in the rat is suitable for use. For example, 5 mg total protein of a test sample is combined with a equal volume of sterile Hydron casting solution, and 5 ml aliquots are pipetted onto the surface of 1 mm Teflon rods glued to the surface of a glass petri dish. Pellets are air-dried in a laminar flow hood (1 hour) and refrigerated overnight. Prior to implantation pellets are rehydrated with a drop of lactated ringers solution.

Animals are anesthetized with metofane and injected with sodium pentobarbital intraperitoneally. A retrobulbar injection of 0.1 ml of 2% lidocaine is made before intracorneal implantation of the Hydron pellet into a surgically created intracorneal pocket approximately 1.5 mm from the limbus. The animals are examined daily with a stereomicroscope. Seven days after implantation, animals are re-anesthetized and perfused sequentially with lactated Ringers solution followed by colloidal carbon. Corneas are harvested, flattened and photographed.

Positive neovascularization responses are recorded only if sustained directional ingrowth of capillary sprouts and hairpin loops towards the implant are observed. Negative responses are recorded when either no growth was observed or when only an occasional sprout or hairpin loop displaying no evidence of sustained growth was detected.

The presence of a non-natural CXCR3 ligand will inhibit the activity of angiogenic agents in the test sample. Thus, a non-natural CXCR3 ligand can be used as a positive control for evaluation of candidate angiogenesis inhibitors in this system. Alternatively, the system can be used to evaluate the activity of a candidate agonist or antagonist of the anti-angiogenic activity of a non-natural CXCR3 ligand.

### Example 4

### Methods for Treating Idiopathic Pulmonary Fibrosis

The present invention provides methods of treating idiopathic pulmonary fibrosis (IPF). The methods generally involve administering to an individual having IPF an effective amount of a non-natural CXCR3 ligand.

In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

An “effective amount” of a non-natural CXCR3 ligand is a dosage that is effective to decrease disease progression by at least about 10%, 20%, 30%, 40%, or even 50% or more, compared with a placebo control or an untreated control.

Disease progression is the occurrence of one or more of the following: (1) a decrease in predicted FVC of 10% or more; (2) an increase in A-a gradient of 5 mm Hg or more; (3) a decrease of 15% of more in single breath DL_{co}. Whether disease progression has occurred is determined by measuring one or more of these parameters on two consecutive occasions 4 to 14 weeks apart, and comparing the value to baseline.

In some embodiments, an “effective amount” of a non-natural CXCR3 ligand is a dosage that is effective to increase progression-free survival time, e.g., the time from baseline (e.g., a time point from 1 day to 28 days before beginning of treatment) to death or disease progression is increased by at least about 10%, 20%, 30%, 40%, or even 50% or more, compared a placebo-treated or an untreated control individual. In some embodiments, an effective amount of a non-natural CXCR3 ligand is a dosage that is effective to increase at least one parameter of lung function, e.g., an effective amount of a non-natural CXCR3 ligand increases at least one parameter of lung function by at least about 10%, 20%, 30%, 40%, or even 50% or more. In some of these embodiments, a determination of whether a parameter of lung function is increased is made by comparing the baseline value with the value at any time point after the beginning of treatment, e.g., 48 weeks after the beginning of treatment, or between two time points, e.g., about 4 to about 14 weeks apart, after the beginning of treatment.

In some embodiments, an effective amount of a non-natural CXCR3 ligand is a dosage that is effective to increase the FVC by at least about 10%, 20%, 30%, 40%, or even 50% or more, compared to baseline on two consecutive occasions 4 to 14 weeks apart.

In some of these embodiments, an effective amount of a non-natural CXCR3 ligand is a dosage that results in a decrease in alveolar:arterial gradient of at least about 5 mm Hg, at least about 7 mm Hg, at least about 10 mm Hg, at least about 12 mm Hg, at least about 15 mm Hg, or more, compared to baseline.

In some of these embodiments, an effective amount of a non-natural CXCR3 ligand is a dosage that increases the single breath DL_{co}, by at least about 10%, 20%, 30%, 40%, or even 50% or more, compared to baseline. DL_{co} is the lung diffusing capacity for carbon monoxide, and is expressed as mL CO/mm Hg/second.

Parameters of lung function include, but are not limited to, forced vital capacity (FVC); forced expiratory volume (FEV₁); total lung capacity; partial pressure of arterial oxygen at rest; partial pressure of arterial oxygen at maximal exertion.

Lung function can be measured using any known method, including, but not limited to spirometry.

### Example 5

### Methods for Treating Liver Fibrosis

The present invention provides methods of treating liver fibrosis, including reducing clinical liver fibrosis, reducing the likelihood that liver fibrosis will occur, and reducing a parameter associated with liver fibrosis. The methods generally involve administering a combination of an effective amount of a subject non-natural CXCR3 ligand to an individual in need thereof. Of particular interest in many embodiments is treatment of humans.

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver.

The present methods generally involve administering a therapeutically effective amount of a subject non-natural CXCR3 ligand. As used herein, an “effective amount” of a subject non-natural CXCR3 ligand is an amount that is effective in reducing liver fibrosis or reduce the rate of progression of liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

The invention also provides a method for treatment of liver fibrosis in an individual comprising administering to the individual an amount of a subject non-natural CXCR3 ligand that is effective for prophylaxis or therapy of liver fibrosis in the individual, e.g., increasing the probability of survival, reducing the risk of death, ameliorating the disease burden or slowing the progression of disease in the individual.

Whether treatment with a subject non-natural CXCR3 ligand is effective in reducing liver fibrosis is determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J Hepatol. 13:372.

The Ishak scoring system is described in Ishak (1995) J. Hepatol. 22:696-699. Stage 0, no fibrosis; stage 1, fibrous expansion of some portal areas, with or without short fibrous septa; stage 2, fibrous expansion of most portal areas, with or without short fibrous septa; stage 3, fibrous expansion of most portal areas with occasional portal to portal (P-P) bridging; stage 4, fibrous expansion of portal areas with marked bridging (P-P) as well as portal-central (P-C); stage 5, marked bridging (P-P and/or P-C) with occasional nodules (incomplete cirrhosis); stage 6, cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a subject non-natural CXCR3 ligand is an amount that effects a change of one unit or more in the fibrosis stage based on pre- and post-therapy liver biopsies. In particular embodiments, a therapeutically effective amount of a subject non-natural CXCR3 ligand reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a subject non-natural CXCR3 ligand. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

In another embodiment, an effective amount of a subject non-natural CXCR3 ligand is an amount that is effective to increase an index of liver function by at least about 10%, 20%, 30%, 40%, or even 50% or more, compared to the index of liver function in an untreated individual, or in a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

In another embodiment, a therapeutically effective amount of a subject non-natural CXCR3 ligand is an amount that is effective to reduce a serum level of a marker of liver fibrosis by at least about 10%, 20%, 30%, 40%, or even 50% or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with a subject non-natural CXCR3 ligand. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

In another embodiment, a therapeutically effective amount of a subject non-natural CXCR3 ligand is an amount that is effective in reducing the incidence (e.g., the likelihood that an individual will develop) of a disorder associated with cirrhosis of the liver by at least about 10%, 20%, 30%, 40%, or even 50% or more, compared to an untreated individual, or in a placebo-treated individual.

Whether combination therapy with a subject non-natural CXCR3 ligand is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective amount of a subject non-natural CXCR3 ligand. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins.

The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another embodiment, a therapeutically effective amount of a subject non-natural CXCR3 ligand is an amount that is effective to increase liver function by at least about 10%, 20%, 30%, 40%, or even 50% or more. For example, a therapeutically effective amount of a subject non-natural CXCR3 ligand is an amount that is effective to reduce an elevated level of a serum marker of liver function by at least about 10%, 20%, 30%, 40%, or even 50% or more, or to reduce the level of the serum marker of liver function to within a normal range. A therapeutically effective amount of a subject non-natural CXCR3 ligand is also an amount effective to increase a reduced level of a serum marker of liver function by at least about 10%, 20%, 30%, 40%, or even 50% or more, or to increase the level of the serum marker of liver function to within a normal range.

### Example 6

### Methods for Treating Renal Fibrosis

Renal fibrosis is characterized by the excessive accumulation of extracellular matrix (ECM) components. Overproduction of transforming growth factor-beta (TGF-β) is believed to underlie tissue fibrosis caused by excess deposition of ECM, resulting in disease. TGF-β's fibrogenic action results from simultaneous stimulation of matrix protein synthesis, inhibition of matrix degradation and enhanced integrin expression that facilitates ECM assembly.

The present invention provides methods of treating renal fibrosis. The methods generally involve administering to an individual having renal fibrosis an effective amount of a subject non-natural CXCR3 ligand. As used herein, an “effective amount” of a subject non-natural CXCR3 ligand that is effective in reducing renal fibrosis; and/or that is effective in reducing the likelihood that an individual will develop renal fibrosis; and/or that is effective in reducing a parameter associated with renal fibrosis; and/or that is effective in reducing a disorder associated with fibrosis of the kidney.

In one embodiment, an effective amount of a subject non-natural CXCR3 ligand is an amount that is sufficient to reduce renal fibrosis, or reduce the rate of progression of renal fibrosis, by at least about 10%, 20%, 30%, 40%, or even 50% or more, compared to the degree of renal fibrosis in the individual prior to treatment, or compared to the rate of progression of renal fibrosis that would have been experienced by the patient in the absence of treatment.

Whether fibrosis is reduced in the kidney is determined using any known method. For example, histochemical analysis of kidney biopsy samples for the extent of ECM deposition and/or fibrosis is performed. Other methods are known in the art. See, e.g., Masseroli et al. (1998) Lab. Invest. 78:511-522; U.S. Pat. No. 6,214,542.

In some embodiments, an effective amount of a subject non-natural CXCR3 ligand is an amount that is effective to increase kidney function by at least about 10%, 20%, 30%, 40%, or even 50% or more, compared to the basal level of kidney function in the individual prior to treatment.

In some embodiments, an effective amount of a subject non-natural CXCR3 ligand is an amount that is effective to slow the decline in kidney function by at least about 10%, 20%, 30%, 40%, or even 50% or more, compared to the decline in kidney function that would occur in the absence of treatment.

Kidney function can be measured using any known assay, including, but not limited to, plasma creatinine level (where normal levels are generally in a range of from about 0.6 to about 1.2 mg/dL); creatinine clearance (where the normal range for creatinine clearance is from about 97 to about 137 mL/minute in men, and from about 88 to about 128 mL/minute in women); the glomerular filtration rate (either calculated or obtained from inulin clearance or other methods), blood urea nitrogen (where the normal range is from about 7 to about 20 mg/dL); and urine protein levels.

In other embodiments, the present invention provides methods that involve administering a synergistic combination of a subject non-natural CXCR3 ligand and a second therapeutic agent. As used herein, a “synergistic combination” of a subject non-natural CXCR3 ligand and a second therapeutic agent is a combined dosage that is more effective in the therapeutic or prophylactic treatment of renal fibrosis than the incremental improvement in treatment outcome that could be predicted or expected from a merely additive combination of (i) the therapeutic or prophylactic benefit of a subject non-natural CXCR3 ligand when administered at that same dosage as a monotherapy and (ii) the therapeutic or prophylactic benefit of the second therapeutic agent when administered at the same dosage as a monotherapy.

The invention also provides a method for treatment of renal fibrosis in an individual comprising administering to the individual a subject non-natural CXCR3 ligand in an amount that is effective for prophylaxis or therapy of renal fibrosis in the individual, e.g., increasing the time to doubling of serum creatinine levels, increasing the time to end-stage renal disease requiring renal replacement therapy (e.g., dialysis or transplant), increasing the probability of survival, reducing the risk of death, ameliorating the disease burden or slowing the progression of disease in the individual.

### Example 7

### Methods for Treating Cancer

The present invention provides methods of treating cancer. The methods generally involve administering an effective amount of a subject non-natural CXCR3 ligand to an individual in need thereof.

The methods are effective to reduce a tumor load by at least about 10%, 20%, 30%, 40%, or even 50% or more, when compared to a suitable control. Thus, in these embodiments, an “effective amount” of a subject non-natural CXCR3 ligand is an amount that is sufficient to reduce tumor load by at least about 10%, 20%, 30%, 40%, or even 50% or more, when compared to a suitable control. In an experimental animal system, a suitable control may be a genetically identical animal not treated with the non-natural CXCR3 ligand. In non-experimental systems, a suitable control may be the tumor load present before administering the non-natural CXCR3 ligand. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

The methods are effective to reduce the growth rate of a tumor by at least about 10%, 20%, 30%, 40%, or even 50% or more, including to total inhibition of growth of the tumor, when compared to a suitable control. Thus, in these embodiments, “effective amounts” of a non-natural CXCR3 ligand is an amount that is sufficient to reduce tumor growth rate by at least about 10%, 20%, 30%, 40%, or even 50% or more, including total inhibition of tumor growth, when compared to a suitable control. In an experimental animal system, a suitable control may be a genetically identical animal not treated with the non-natural CXCR3 ligand. In non-experimental systems, a suitable control may be the tumor load present before administering the non-natural CXCR3 ligand. Other suitable controls may be a placebo control.

Whether growth of a tumor is inhibited can be determined using any known method, including, but not limited to, an in vitro proliferation assay; a ³H-thymidine uptake assay; and the like.

The methods are useful for treating a wide variety of cancers, including carcinomas, sarcomas, leukemias, and lymphomas. Particular types of these cancers are disclosed in International Patent Application WO 2005/016241, which is, herein, incorporated by reference.

### Example 8

### Methods for Treating Angiogenic Disorders

The present invention provides methods for treating angiogenic disorders. The methods generally involve administering an effective amount of a subject non-natural CXCR3 ligand to an individual in need thereof.

In a subject method of treating an angiogenic disorder, an “effective amount” of a subject non-natural CXCR3 ligand is an amount that is angiostatic, e.g., an amount that reduces angiogenesis by at least about 10%, 20%, 30%, 40%, or even 50% or more, compared with the level of angiogenesis in the absence of treatment with the non-natural CXCR3 ligand.

Many systems are available for assessing angiogenesis. For example, as angiogenesis is required for solid tumor growth, the inhibition of tumor growth in an animal model may be used as an index of the inhibition of angiogenesis. Angiogenesis may also be assessed in terms of models of wound-healing, in cutaneous or organ wound repair; and in chronic inflammation, e.g., in diseases such as rheumatoid arthritis, atherosclerosis and idiopathic pulmonary fibrosis (IPF). It may also be assessed by counting vessels in tissue sections, e.g., following staining for marker molecules, e.g., CD3H, Factor VIII, or PECAM-1.

Whether angiogenesis is reduced can be determined using any method known in the art, including, e.g., stimulation of neovascularization into implants impregnated with relaxin; stimulation of blood vessel growth in the cornea or anterior eye chamber; stimulation of endothelial cell proliferation, migration or tube formation in vitro; and the chick chorioallantoic membrane assay; the hamster cheek pouch assay; the polyvinyl alcohol sponge disk assay. Such assays are well known in the art and have been described in numerous publications, including, e.g., Auerbach et al. ((1991) Pharmac. Ther. 51:1-11), and references cited therein.

A system in widespread use for assessing angiogenesis is the corneal micropocket assay of neovascularization, as may be practiced using rat corneas. This in vivo model is widely accepted as being generally predictive of clinical usefulness. See, e.g., O'Reilly et. al. (1994) Cell 79:315-328, Li et. al. (1991) Invest. Ophthalmol. Vis. Sci. 32(11):2898-905; and Miller et. al. (1994) Am. J. Pathol. 145(3):574-84.

### Example 9

Human microvascular endothelial cells of the lung (HMVEC), human aortic endothelial cells (HUVEC) and murine pre-B-cells expressing stably transfected human CXCR3 were cultured in defined medium, supplemented with serum and growth factors. Prior to assay, the cells were serum-starved overnight, and stimulated with a panel of chemokines for up to 15 minutes: recombinant human IP-10, I-TAC, MIG and PF4 (R&D Systems, catalog #s 266-IP/CF, 672-IP/CF, 392-MG/CF and 795-P4/CF, respectively); consensus I-TAC ligand (SEQ ID NO: 2); consensus I-TAC ligand with native I-TAC N-loop (SEQ ID NO: 3); consensus IP-10 ligand (SEQ ID NO: 5); consensus IP-10 ligand with native I-TAC N-loop (SEQ ID NO: 6); consensus MIG ligand (SEQ ID NO: 8); consensus MIG ligand with native I-TAC N-loop (SEQ ID NO: 9); and native PF4 with native I-TAC N-loop (SEQ ID NO: 13).

Cellular lysates were prepared and phosphorylation of ERK1/2 was determined via immunoblotting after SDS-PAGE to show activation of the CXCR3 receptor. Non-phosphorylated ERK1/2 was also determined via immunoblotting and used as a loading control. The final images were scanned, the band intensities digitized and the phosphorylated ERK1/2 bands were normalized to the loading controls. The results are summarized as fold induction over control levels in the following table.

| | HMVEC | HUVEC | Pre-B-cells |
|---|---|---|---|
| Control | 1.000 | 1.000 | 1.000 |
| Rh-I-TAC, 2 min. | 0.205 | 1.646 | 2.244 |
| Rh-I-TAC, 5 min. | 0.137 | 1.356 | 1.376 |
| Rh-I-TAC, 15 min. | 3.077 | 1.310 | 1.223 |
| c-I-TAC, 2 min. | 0.283 | 1.182 | 0.897 |
| c-I-TAC, 5 min. | 0.132 | 1.325 | 1.333 |
| c-I-TAC, 15 min. | 2.045 | 1.270 | 1.423 |
| N-I-TAC, 2 min. | 0.275 | 1.269 | 2.164 |
| N-I-TAC, 5 min. | 0.296 | 1.571 | 1.892 |
| N-I-TAC, 15 min. | 4.771 | 1.385 | 0.337 |
| Rh-IP10, 2 min. | 0.333 | 10.300 | 1.034 |
| Rh-IP10, 5 min. | 0.070 | 10.831 | 0.921 |
| Rh-IP10, 15 min. | 0.382 | 6.866 | 0.570 |
| c-IP10, 2 min. | 0.098 | 4.109 | 0.674 |
| c-IP10, 5 min. | 0.180 | 5.730 | 0.765 |
| c-IP10, 15 min. | 3.935 | 5.598 | 0.722 |
| N-IP10, 2 min. | 0.194 | 3.204 | 0.690 |
| N-IP10, 5 min. | 0.341 | 6.559 | 1.040 |
| N-IP10, 15 min. | 4.551 | 4.935 | 0.829 |
| Rh-MIG, 2 min. | 1.564 | 5.347 | 0.795 |
| Rh-MIG, 5 min. | 1.131 | 6.959 | 0.842 |
| Rh-MIG, 15 min. | 1.313 | 5.765 | 1.756 |
| c-MIG, 2 min. | 1.058 | 2.287 | 1.071 |
| c-MIG, 5 min. | 1.344 | 3.342 | 1.918 |
| c-MIG, 15 min. | 1.624 | 6.863 | 2.113 |
| N-MIG, 2 min. | 1.163 | 2.294 | 0.986 |
| N-MIG, 5 min. | 0.899 | 6.577 | 1.024 |
| N-MIG, 15 min. | 2.327 | 6.950 | 2.178 |
| Rh-PF4, 2 min. | 1.014 | 6.854 | 1.253 |
| Rh-PF4, 5 min. | 0.895 | 9.627 | 1.328 |
| Rh-PF4, 15 min. | 1.782 | 11.516 | 1.446 |
| N-PF4, 2 min. | 0.915 | 4.134 | 1.575 |
| N-PF4, 5 min. | 1.058 | 5.679 | 1.157 |
| N-PF4, 15 min. | 3.278 | 9.832 | 0.931 |

One skilled in the art will recognize that the above examples are provided to illustrate the invention and should in no way limit the scope of the invention.

All patents and publications in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A non-natural CXCR3 polypeptide receptor ligand wherein the N-loop domain is from iTAC.

2. The non-natural CXCR3 polypeptide receptor ligand of claim 1 wherein said polypeptide receptor ligand except for the N-loop domain has a native amino acid sequence.

3. The non-natural CXCR3 polypeptide receptor ligand of claim 1 wherein said polypeptide receptor ligand except for the N-loop domain has native amino acid sequence and non-native amino acid sequence, wherein said non-native amino acid sequence results from substituting at least one native amino acid with an amino acid from a homologous position of a different CXCR3 polypeptide receptor ligand.

4. The non-natural CXCR3 polypeptide receptor ligand of claim 3 wherein said amino acid from a homologous position of a different CXCR3 polypeptide receptor ligand is a consensus amino acid residue.

5. A non-natural CXCR3 polypeptide ligand comprising a sequence selected from the group consisting of SEQ ID NO: 3, SEQ ID NO: 6, SEQ ID NO: 9, SEQ ID NO: 10, and SEQ ID NO: 11, and variations, thereof, wherein positions other than 12-17 are changed to an amino acid that taken from a homologous position in any of iTAC, IP-10 or MIG CXCR3 polypeptide ligands.

6. A non-natural PF4 CXCR3 polypeptide receptor ligand wherein the N-loop domain is from iTAC.

7. The non-natural PF4 CXCR3 polypeptide receptor ligand of claim 6 wherein said polypeptide receptor ligand except for the N-loop domain has a native amino acid sequence of PF4.

8. The non-natural PF4 CXCR3 polypeptide receptor ligand of claim 7 having a polypeptide sequence comprising SEQ ID NO: 13.

9. The non-natural CXCR3 polypeptide receptor ligand of any of claims 1-5 wherein the receptor ligand is a mature form of the ligand and includes an N-terminal methionine.

10. The non-natural PF4 CXCR3 polypeptide receptor ligand of any of claims 6-8 wherein the receptor ligand is a mature form of the ligand and includes an N-terminal methionine.

11. The non-natural CXCR3 polypeptide receptor ligand of any of claims 1-5 or 9, wherein one or more amino acids of the polypeptide are chemically modified.

12. The non-natural PF4 CXCR3 polypeptide receptor ligand of any of claims 6-8 or 10, wherein one or more amino acids of the polypeptide are chemically modified.

13. The non-natural CXCR3 polypeptide receptor ligand of claim 11, wherein the non-natural CXCR3 polypeptide receptor ligand is modified by pegylation.

14. The non-natural PF4 CXCR3 polypeptide receptor ligand of claim 12, wherein the non-natural PF4 CXCR3 polypeptide receptor ligand is modified by pegylation.

15. The non-natural CXCR3 polypeptide receptor ligand of any of claims 1-5, 9, or 11, wherein the non-natural CXCR3 polypeptide receptor ligand is a fusion polypeptide.

16. The non-natural PF4 CXCR3 polypeptide receptor ligand of any of claims 6-8, 10, or 12, wherein the non-natural PF4 CXCR3 polypeptide receptor ligand is a fusion polypeptide.

17. A polynucleotide comprising a sequence encoding the non-natural CXCR3 polypeptide receptor ligand of any of claims 1-16.

18. An expression vector comprising the polynucleotide of claim 17 operably linked to a promoter.

19. A viral expression vector comprising the polynucleotide of claim 17.

20. A host cell comprising the polynucleotide of claim 17 or 18.

21. A host cell comprising the expression vector of claim 18.

22. A method for producing a non-natural CXCR3 ligand, the method comprising culturing the host cell of claim 21 under conditions that favor production of the non-natural CXCR3 ligand and isolating the non-natural CXCR3 ligand from the culture.

23. An antibody that specifically binds a non-natural CXCR3 ligand of any of claims 1-16.

24. A method for treating a fibrotic disease in an individual, the method comprising administering to an individual suffering from a fibrotic disease an amount of a non-natural CXCR3 ligand of any of claims 1-16 that is effective in the treatment or prophylaxis of the fibrotic disease in the individual.

25. A method for treating a fibrotic disease in an individual, the method comprising administering to an individual suffering from a fibrotic disease an amount of a polynucleotide encoding a non-natural CXCR3 ligand of any of claims 1-16 that is effective in the treatment or prophylaxis of the fibrotic disease in the individual.

26. The method of claim 25, wherein the polynucleotide encoding the non-natural CXCR3 ligand is provided in a viral vector.

27. The method of any of claims 24-26, wherein the fibrotic disease is pulmonary fibrosis.

28. The method of claim 27, wherein the pulmonary fibrosis is idiopathic pulmonary fibrosis.

29. The method of claim 27, wherein the pulmonary fibrosis is from a known etiology.

30. The method of claim 27, wherein the fibrotic disease is selected from liver fibrosis, renal fibrosis, cardiac fibrosis, and scleroderma.

31. A method of reducing tumor growth in an individual having a tumor, the method comprising administering to the individual an effective amount of a non-natural CXCR3 ligand of any of claims 1-16.

32. A method for reducing tumor growth in an individual, the method comprising administering to an individual suffering from a fibrotic disease an amount of a polynucleotide encoding a non-natural CXCR3 ligand of any of claims 1-16 that is effective in the treatment or prophylaxis of the fibrotic disease in the individual.

33. The method of claim 32, wherein the polynucleotide encoding the non-natural CXCR3 ligand is provided in a viral vector.

34. The method of any of claims 24-33, further comprising administering an effective amount of an anti-neoplastic agent selected from an alkylating agent, a nitrosourea, an antimetabolite, an antitumor antibiotic, a plant (vinca) alkaloid, a taxane, and a steroid hormone.

35. The method of any of claims 24-34, wherein the individual is a human.
